(19) United States
(12) Patent Application Publication
(10) Pub. No.: US 2012/0184791 A1
(43) Pub. Date: Jul. 19, 2012

(54) PROCESS FOR THE MANUFACTURE OF A FORMULATED OXYGENATE CONVERSION CATALYST, FORMULATED OXYGENATE CONVERSION CATALYST AND PROCESS FOR THE PREPARATION OF AN OLEFINIC PRODUCT

(76) Inventors: Leslie Andrew Chewter, Amsterdam (NL); Jeroen Van Westrenen, Amsterdam (NL); Ferry Winter, Amsterdam (NL)

(21) Appl. No.: 13/320,962

(22) PCT Filed: Apr. 29, 2010
(86) PCT No.: PCT/EP10/55824

§ 371 Date: Dec. 21, 2011

(30) Foreign Application Priority Data
May 19, 2009 (EP) 09160635.0

(51) Int. Cl. C07C 1/20; B01J 27/182

(52) U.S. Cl. 585/638; 502/214; 502/162

(57) ABSTRACT

The present invention provides a process for the manufacture of a formulated oxygenate conversion catalyst, the process comprising combining a first molecular sieve comprising aluminosilicate, a second molecular sieve, different from the first molecular sieve, the second molecular sieve having more-dimensional channels, and a matrix material; and, treating the catalyst with a phosphorus containing compound after combination of the molecular sieves with the matrix material. In a further aspect the invention provides a formulated oxygenate conversion catalyst and a process for the preparation of an olefinic product.

This invention relates to a process for the manufacture of an oxygenate conversion catalyst, an formulated oxygenate conversion catalyst and a process for the preparation of an olefin product.

Processes for the preparation of olefins from oxygenates are known in the art. Of particular interest is often the production of light olefins, in particular ethylene and/or propylene. The oxygenate feedstock can for example comprise methanol and/or dimethyl ether, and an interesting route includes their production from synthesis gas derived from e.g. natural gas or via coal gasification.

For example, WO2007/135052 discloses a process wherein an alcohol and/or ether containing oxygenate feedstock and an olefinic co-feed are reacted in the presence of a zeolite having one-dimensional 10-membered ring channels to prepare an olefinic reaction mixture, and wherein part of the obtained olefinic reaction mixture is recycled as olefinic co-feed. With a methanol and/or dimethyl ether containing feedstock, and an olefinic co-feed comprising C4 and/or C5 olefins, an olefinic product rich in light olefins can be obtained.

Various by-products are normally formed in the oxygenate to olefin reaction, such as aromatics and saturated hydrocarbons. In some cases this results in uneconomical streams being produced and for certain by-products the catalysts may be coked and deactivated. Thus the saturates make and aromatic make of an oxygenate to olefins reaction is preferably minimised.

U.S. Pat. No. 4,579,994 discloses the treatment of pure ZSM-5 zeolite to incorporate therein, inter alia, a phosphorus-containing compound, for a conversion reaction of methanol to olefins. This disclosure teaches to treat the zeolite as such, and mentions that the treated zeolite catalyst can be mixed with a carrier such as clay, kaolin and alumina. This is not a useful method for manufacturing spray-dried catalyst particles having good attrition resistance using a silica binder. Such spray-dried particles are prepared by mixing a molecular sieve component with, inter alia, a silica binder, and requires an ion exchange after spray drying to remove the alkaline used for the preparation of the binder from the catalyst. Therefore, phosphorus previously incorporated in the zeolite may fully or partially be lost during this ion exchange. A similar process is disclosed in U.S. Pat. No. 6,797,851.

There is a need for an improved and efficient oxygenate-to-olefins process, wherein a minimum of by-products is formed.

According to a first aspect of the present invention there is provided a process for the manufacture of a formulated oxygenate conversion catalyst, the process comprising combining a first molecular sieve comprising aluminosilicate, a second molecular sieve, different from the first molecular sieve, the second molecular sieve having more-dimensional channels, and a matrix material; and,
treating the catalyst with a phosphorus containing compound after combination of the molecular sieves with the matrix material, preferably wherein the phosphorus containing compound is not a Group II metal phosphate.

In a second aspect the invention provides a formulated oxygenate conversion catalyst obtainable by the process according to the first aspect of the invention, comprising:
a first molecular sieve comprising aluminosilicate;
a second molecular sieve, different from the first molecular sieve, the second molecular sieve having more-dimensional channels;
a matrix material;
wherein the catalyst comprises more of the first molecular sieve, than of the second molecular sieve, based on weight;
and wherein the formulated oxygenate conversion catalyst comprises a phosphorus or a phosphorus containing compound, preferably wherein the phosphorus containing compound is not a Group II metal phosphate.

The catalyst is treated with a phosphorus-containing compound after the synthesis thereof, i.e. after the combination of molecular sieves and matrix. The treatment typically leads to a deposition of phosphorus species. The phosphorus may be present in an amount of from 0.05 to 10 wt % of the total catalyst (formulated catalyst), preferably of from 0.05 to 5 wt %, more preferably of from 0.2-2.5 wt %, especially 1-2 wt %. The catalyst is post-treated, i.e. the phosphorus treatment takes place after the combination of molecular sieves and matrix, but it is also possible to perform a phosphorus treatment of at least one molecular sieve before the combination. Before or after phosphorus treatment a calcination can take place.

Preferably the phosphorus-containing compound comprises a phosphorus species such as PO₄³⁻, P—(OCH₃)₃, or P₂O₅, especially PO₄³⁻. Preferably the phosphorus-containing compound comprises a compound selected from the group consisting of ammonium phosphate, ammonium dihydrogen phosphate, dimethylphosphate, metaphosphoric acid and trimethyl phosphite and phosphoric acid, especially phosphoric acid.

Preferably the phosphorus containing compound is not a Group II metal phosphate. Group II metal species include magnesium, calcium, strontium and barium; especially calcium.

The external surface area of the formulated catalyst is normally 1-500 m²/g, preferably 40-200 m²/g. “External surface area” as used herein refers to the total surface area of the formulated catalyst excluding the surface area of micropores. Micropores are defined herein as pores with widths not exceeding 2.0 nm.

The expression ‘molecular sieve’ is used in the description and claims for a material containing small regular pores and/or channels and exhibiting catalytic activity in the conversion of oxygenate to olefin. Where reference is made in the description and in the claims to a molecular sieve, this can in particular be a zeolite. A zeolite is understood to be an aluminosilicate molecular sieve, also referred to as aluminosilicate. The first molecular sieve is an aluminosilicate and preferably has one-dimensional 10-membered ring channels. These are understood to be aluminosilicates having only 10-membered ring channels in one direction which are not intersected by other 8, 10 or 12-membered ring channels from another direction.

Preferably, the first molecular sieve is selected from the group of TON-type (for example zeolite ZSM-22), MTT-type (for example zeolite ZSM-23), STF-type (for example SSZ-35), SFF-type (for example SSZ-44), EUO-type (for example ZSM-50), and EU-2-type aluminosilicates or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type aluminosilicates are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type aluminosilicates are more particularly described in e.g. U.S. Pat. No. 4,397,827. For purposes of the present invention, EU-2 is considered to include its isotypes, e.g., ZSM-48. In a further preferred embodiment an aluminosilicate of the MTT-type, such as ZSM-23, and/or a TON-type, such as ZSM-22 is used as the first molecular sieve.

Molecular sieve and zeolite types are for example defined in Ch. Baerlocher and L. B. McCusker, Database of Zeolite Structures: http://www.iza-structure.org/databases/, which database was designed and implemented on behalf of the Structure Commission of the International Zeolite Association (IZA-SC), and based on the data of the 4th edition of the Atlas of Zeolite Structure Types (W. M. Meier, D. H. Olson and Ch. Baerlocher). The Atlas of Zeolite Framework Types, 5th revised edition 2001 and 6^{th} edition 2007 may also be consulted.

In one embodiment, aluminosilicates in the hydrogen form are used in the oxygenate conversion catalyst particles, e.g., HZSM-22, HZSM-23, and HZSM-48, HZSM-5. Preferably at least 50% w/w, more preferably at least 90% w/w, still more preferably at least 95% w/w and most preferably 100% of the total amount of aluminosilicate used is in the hydrogen form. When the aluminosilicates are prepared in the presence of organic cations the aluminosilicate may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 300° C. The aluminosilicates obtained after ion-exchange are also referred to as being in the ammonium form.

Where a molecular sieve having one-dimensional 10-membered ring channels is used, preferably it has a silica to alumina ratio (SAR) in the range of from 1 to 500. A particularly suitable SAR is less than 200, in particular 150 or less. A preferred range is from 10 to 200 or from 10-150. The SAR is defined as the molar ratio of SiO₂/Al₂O₃ corresponding to the composition of the aluminosilicate.

For ZSM-22, a SAR in the range of 40-150 is preferred, in particular in the range of 50-140, more in particular 70-120. Good performance in terms of activity and selectivity has been observed with a SAR of about 100.

For ZSM-23, a SAR in the range of 20-120 is preferred, in particular in the range of 30-80. Good performance in terms of activity and selectivity has been observed with a SAR of about 50.

Typically the formulated catalyst comprises catalyst particles, and preferably the individual catalyst particles comprise both the first molecular sieve and the second molecular sieve.

Thus typically the first and second molecular sieves are intimately mixed, that is crystals of the first and second molecular sieves are present in the same particle, rather than a mixture of formulated catalyst particles where individual particles have one or other molecular sieves, not both. Preferably therefore an average distance between a crystal of the first molecular sieve and a crystal of the second molecular sieve is less than an average particle size of the catalyst particles, preferably 40 μm or less, more preferably 20 μm or less, especially 10 μm or less. For near-spherical particles the average particle size can be determined by the weight-averaged diameter of a statistically representative quantity of particles, such as of e.g. 10 mg, 100 mg, 250 mg, or 1 g of particles. Such a statistically representative quantity of particles is referred to herein as a bed of particles. For other shapes of catalyst particles the skilled person knows how to define a suitable average of a characteristic dimension as average particle size, preferably a weight-average is used. The average distance between a crystal of the first molecular sieve and a crystal of the second molecular sieve can be determined using for instance electron microscopy.

An intimate mix the first and second molecular sieves is for example obtained when a mixture comprising the first and second molecular sieves and matrix are spray dried to form the catalyst particles. Typically a mixture comprising the first and second molecular sieves are milled, either separately but preferably together, before the matrix is added.

Alternatively the first and second molecular sieves may be co-crystallised or intergrown in order to form intimately mixed catalyst particles. For such embodiments a matrix is typically added after co-crystallisation and the resulting mixture then spray dried. Co-crystallisation and intergrowth of two or more molecular sieves are well known processes to the skilled person and does not need any further explanation.

Preferably, such amounts of first and second molecular sieve are combined that the formulated oxygenate conversion catalyst comprises at least 1 wt %, based on total molecular sieve in the oxygenate conversion catalyst particles, of the second molecular sieve having more-dimensional channels, in particular at least 5 wt %, more in particular at least 8 wt %; based on total molecular sieve content. The presence of a minority portion of a molecular sieve having more-dimensional channels in the oxygenate conversion catalyst particles was found to improve stability (slower deactivation during extended runs) and hydrothermal stability. Without wishing to be bound by a particular hypothesis or theory, it is presently believed that this is due to the possibility for converting larger molecules by the molecular sieve having more-dimensional channels, that were produced by the aluminosilicate having one-dimensional 10-membered ring channels, and which would otherwise form coke.

The molecular sieve having more-dimensional channels is understood to have intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. The more-dimensional molecular sieve can be for example a FER type zeolite which is a two-dimensional structure and has 8- and 10-membered rings intersecting each other. Preferably however the intersecting channels in the more-dimensional molecular sieve are each 10-membered ring channels. Thus the more-dimensional molecular sieve may be a zeolite, or a SAPO-type (silicoaluminophosphate) molecular sieve. More preferably however the more-dimensional molecular sieve is a zeolite. A suitable more-dimensional molecular sieve is an MFI-type zeolite, in particular zeolite ZSM-5. Another suitable more-dimensional molecular sieve is a MEL-type aluminosilicate, in particular zeolite ZSM-11. The weight ratio between the aluminosilicate having one-dimensional 10-membered ring channels, and the second molecular sieve having more-dimensional channels can be in the range of from 1:100 to 100:1, preferably 1:1 to 100:1, more preferably in the range of 9:1 to 2:1.

Preferably the molecular sieve having more-dimensional channels has a silica-to-alumina ratio (SAR) in the range from 1 to 1000. For ZSM-5, a SAR of 60 or higher is preferred, in particular 80 or higher, more preferably 100 or higher, still more preferably 150 or higher, such as 200 or higher. At higher SAR the percentage of C4 saturates in the C4 totals produced is minimized.

In special embodiments the oxygenate conversion catalyst particles can comprise less than 35 wt % of the second molecular sieve, based on the total molecular sieve in the oxygenate conversion catalyst particles, in particular less than 20 wt %, more in particular less than 18 wt %, still more in particular less than 15 wt %.

In one embodiment the oxygenate conversion catalyst particles can comprise more than 50 wt %, at least 65 wt %, based on total molecular sieve in the oxygenate conversion catalyst particles, of the aluminosilicate having one-dimensional 10-membered ring channels. The presence of a majority of such aluminosilicate strongly determines the predominant reaction pathway.

The aluminosilicate is used in a formulation, i.e. within the matrix material. For the purposes of this invention ‘matrix’ is herein defined as including any active matrix component as well as any filler and/or binder. Other components can also be present in the formulation. In a formulation, the aluminosilicate in combination with the matrix such as binder and/or filler material is/are also referred to as a formulated oxygenate conversion catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the aluminosilicate is therefore incorporated in a binder material. Examples of suitable materials in a formulation include active and inactive materials and synthetic or naturally occurring zeolites as well as inorganic materials such as clays, silica, alumina, silica-alumina, titania, zirconia and aluminosilicate. For present purposes, inert materials, such as silica, are preferred because they may prevent unwanted side reactions which may take place in case a more acidic material, such as alumina or silica-alumina is used.

The matrix material may be selected from the group consisting of: silica, magnesia, titania, kaolin, montmorillonite; preferably kaolin. Where kaolin is used, preferably it has less than 3 wt %, preferably less than 1.5 wt % iron, and preferably less than 4 wt %, preferably less than 3 wt % titania; all based on total content of the kaolin.

The skilled artisan knows that silica binders can be prepared at low and high pH stabilized by alkaline (Na⁺), ammonium (NH₄⁺) and/or by acid (H⁺). A silica binder that is useful for obtaining spray dried catalyst with good attrition resistance, the binder is stabilized at very low pH (<1.5) or with high alkaline content. High alkaline is preferred, since low pH stabilization may influence the molecular sieve in such environment.

The oxygenate conversion catalyst particles preferably have an average particle size of less than 100 microns.

Preferably the catalyst is treated with the phosphorus containing compound by impregnation.

In contrast to U.S. Pat. No. 4,579,994 or for instance U.S. Pat. No. 6,797,851, the inventors of the present invention have found that by adding a phosphorus-containing compound to a formulated catalyst rather than the aluminosilicate as such, that beneficial results may be obtained.

The formulated catalyst is preferably produced by spray-drying a slurry of the aluminosilicate and matrix then drying and typically calcining. Typically the spray drying step is performed before addition of the phosphorus-containing compound.

A calcining step may be performed before and after addition of the phosphorus-containing compound.

Calcining is herein defined as heating the catalyst to a temperature of above 250° C., preferably above 350° C., for at least 30 minutes, preferably at least 4 hours, optionally in the presence of an inert gas and/or oxygen and/or steam. The phosphorus containing compound can be converted to a final phosphorus species on the catalyst during calcination. The phosphorus species on the catalyst is preferably an inorganic phosphorus species. E.g., P₂O₅ can be formed.

The phosphorus-containing compound preferably comprises a phosphate. The phosphorus-containing compound may be a compound chosen from the group consisting of ammonium phosphate, ammonium dihydrogen phosphate, dimethylphosphate, metaphosphoric acid and trimethyl phosphite. Typically the phosphorus-containing compound is water soluble.

Preferably the phosphorus-containing compound is impregnated into the catalyst. During impregnation, a predetermined amount of a solution, such as an aqueous solution, of the phosphorus-containing compound is blended with a predetermined quantity of catalyst. After evaporation of the solvent, a controlled amount of the phosphor compound is left on the catalyst.

Accordingly, the present invention in one aspect provides a formulated oxygenate conversion catalyst obtainable by impregnating a catalyst composition comprising a first molecular sieve comprising aluminosilicate; a second molecular sieve, different from the first molecular sieve, the second molecular sieve having more-dimensional channels; and a matrix material; with a phosphorus containing compound.

Typically the catalyst particles are calcined after addition of the phosphorus-containing compound and a phosphate species remains on the catalyst particles, especially at an acidic site on the aluminosilicate.

Without wishing to be bound by a particular hypothesis or theory, it is considered that the outer surface of the molecular sieve(s) and surface at the entrance of the channels has poor selectivity to the intended olefinic product, whereas channels in the molecular sieve(s) have better selectivity towards the intended olefinic product, and it is considered that the phosphorus treatment according to the present invention preferentially inhibits the activity of the outer surface of the molecular sieve(s) compared to the channels in the molecular sieves. Acid sites on the outer surface and acid sites at the entrance of the channels of the molecular sieve are thought to be a cause of unwanted by-product formation. This is particularly pronounced for molecular sieves present as relatively small crystals therefore having a relatively large surface-to-volume ratio. In a catalyst formulation including a molecular sieve with one-dimensional 10-membered ring channels and a more-dimensional molecular sieve it is believed that there are more unwanted by-product reactions caused by the more-dimensional molecular sieve. One factor can be that the more-dimensional molecular sieve is typically present as similar or smaller crystals than the one-dimensional molecular sieve, i.e. has a higher surface-to-volume ratio. It is presently believed that the present invention particularly passivates outer surface acid sites and the acid sites at the entrance of the channels on the more-dimensional molecular sieve component, such as of MFI- or MEL-type.

The formulated catalyst according to the invention is particularly useful to catalyse the preparation of an olefinic product from an oxygenate feedstock, the process comprising reacting an oxygenate feedstock in the presence of formulated oxygenate conversion catalyst particles to produce an olefinic product. Thus according to a further aspect the invention provides a process for the preparation of an olefinic product in the presence of the catalyst of the invention, the process comprising reacting an oxygenate feedstock in the presence of the catalyst according to the second aspect of the invention to produce the olefinic product, in particular in the presence of an olefinic co-feed.

The oxygenate feedstock comprises oxygenate species having an oxygen-bonded methyl group, such as methanol, dimethyl ether. Preferably the oxygenate feedstock comprises at least 50 wt % of methanol and/or dimethyl ether, more preferably at least 80 wt %, most preferably at least 90 wt %.

The oxygenate feedstock can be obtained from a different or separate reactor, which converts methanol at least partially into dimethyl ether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of water, preferably less than 10 wt %, more preferably less than 5 wt %. Preferably the oxygenate feedstock contains essentially no hydrocarbons other than oxygenates, i.e. less than 5 wt %, preferably less than 1 wt %.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

Preferably the oxygenate feedstock is reacted to produce the olefinic product in the presence of an olefinic co-feed. By an olefinic composition or stream, such as an olefinic product, product fraction, fraction, effluent, reaction effluent or the like is understood a composition or stream comprising one or more olefins, unless specifically indicated otherwise. Other species can be present as well. Apart from olefins, the olefinic co-feed may contain other hydrocarbon compounds, such as for example paraffinic compounds. Preferably the olefinic co-feed comprises an olefinic portion of more than 50 wt %, more preferably more than 60 wt %, for example more than 70 wt %, which olefinic portion consists of olefin(s). The olefinic co-feed can also consist essentially of olefin(s).

Any non-olefinic compounds in the olefinic co-feed are preferably paraffinic compounds. Such paraffinic compounds are preferably present in an amount in the range of from 0 to 50 wt %, more preferably in the range of from 0 to 40 wt %, still more preferably in the range of from 0 to 30 wt %.

By an olefin is understood an organic compound containing at least two carbon atoms connected by a double bond. The olefin can be a mono-olefin, having one double bond, or a poly-olefin, having two or more double bonds. Preferably olefins present in the olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferably the olefinic co-feed is at least partially obtained by a recycle stream formed by recycling a suitable fraction of the reaction product comprising C4 olefin. The skilled artisan knows how to obtain such fractions from the olefinic reaction effluent such as by distillation.

In one embodiment at least 70 wt % of the olefinic co-feed, during normal operation, is formed by the recycle stream, preferably at least 90 wt %, more preferably at least 99 wt %. Most preferably the olefinic co-feed is during normal operation formed by the recycle stream, so that the process converts oxygenate feedstock to predominantly light olefins without the need for an external olefins stream. During normal operation means for example in the course of a continuous operation of the process, for at least 70% of the time on stream. The olefinic co-feed may need to be obtained from an external source, such as from a catalytic cracking unit or from a naphtha cracker, during start-up of the process, when the reaction effluent comprises no or insufficient C4+ olefins.

The C4 fraction contains C4 olefin(s), but can also contain a significant amount of other C4 hydrocarbon species, in particular C4 paraffins, because it is difficult to economically separate C4 olefins and paraffins, such as by distillation.

In one embodiment the olefinic co-feed and preferably also the recycle stream comprises C4 olefins and less than 10 wt % of C5+ hydrocarbon species, more preferably at least 50 wt % of C4 olefins, and at least a total of 70 wt % of C4 hydrocarbon species.

The olefinic co-feed and preferably also the recycle stream, can in particular contain at least a total of 90 wt % of C4 hydrocarbon species. In one embodiment, the olefinic co-feed comprises less than 5 wt % of C5+ olefins, preferably less than 2 wt % of C5+ olefins, even more preferably less than 1 wt % of C5+ olefins, and likewise the recycle stream. In another embodiment, the olefinic co-feed, comprises less than 5 wt % of C5+ hydrocarbon species, preferably less than 2 wt % of C5+ hydrocarbon species even more preferably less than 1 wt % of C5+ hydrocarbon species, and likewise the recycle stream.

Thus in certain preferred embodiments, the olefinic portion of the olefinic co-feed, and of the recycle stream, comprises at least 90 wt % of C4 olefins, more preferably at least 99 wt %. Butenes as co-feed have been found to be particularly beneficial for high ethylene selectivity. Therefore one particularly suitable recycle stream consists essentially, i.e. for at least 99 wt %, of 1-butene, 2-butene (cis and trans), isobutene, n-butane, isobutane, butadiene.

In further embodiments the recycle stream can contain a larger fraction of C5 and/or higher olefins. It is for example possible to recycle more than 50% or substantially all of the C5 olefins in the reactor effluent.

In certain embodiments, the recycle stream can also comprise propylene. This may be preferred when a particularly high production of ethylene is desired, so that part or all of the propylene produced is recycled together with C4 olefins.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 20:1 to 1:10, more preferably in the range of 15:1 to 1:5.

In a preferred embodiment wherein the oxygenate comprises only one oxygen-bonded methyl group, such as methanol, the molar ratio preferably lies in the range of from 20:1 to 1:5 and more preferably in the range of 15:1 to 1:2.5.

In another preferred embodiment wherein the oxygenate comprises two oxygen-bonded methyl groups, such as for example dimethylether, the molar ratio preferably lies in the range from 10:1 to 1:10.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed, if used. Such olefins may for example be obtained from a steam cracker, a catalytic cracker, alkane dehydrogenation (e.g. propane or butane dehydrogenation). Further, such olefins can be bought from the market.

In a special embodiment the olefins for such start-up are obtained from a previous process that converted oxygenates, with or without olefinic co-feed, to olefins. Such a previous process may have been located at a different location or it may have been carried out at an earlier point in time.

Since a molecular sieve having more-dimensional channels such as ZSM-5 is present in the oxygenate conversion catalyst particles, even in minority compared to the first molecular sieve, start up is possible without an olefinic co-feed from an external source. ZSM-5 for example is able to convert an oxygenate to an olefin-containing product, so that a recycle can be established.

Typically the oxygenate conversion catalyst particles deactivate in the course of the process. Conventional catalyst regeneration techniques can be employed, such as burning of coke in a regenerator. The formulated catalyst used in the process of the present invention can have any shape known to the skilled person to be suitable for this purpose, for it can be present in the form of spray-dried particles, spheres, tablets, rings, extrudates, etc. Extruded catalysts can be applied in various shapes, such as, cylinders and trilobes. If desired, spent oxygenate conversion catalyst particles can be regenerated and recycled to the process of the invention. Spray-dried particles allowing use in a fluidized bed or riser reactor system are preferred.

Spherical particles are normally obtained by spray drying. Preferably the average particle size is in the range of 1-200 μm, preferably 50-100 μm.

The reactor system used to produce the olefins may be any reactor known to the skilled person and may for example contain a fixed bed, moving bed, fluidized bed, riser reactor and the like. In one embodiment a riser reactor system can be used, in particular a riser reactor system comprising a plurality of serially arranged riser reactors. In another embodiment, a fast fluidized bed reactor can be used.

The reaction to produce the olefins can be carried out over a wide range of temperatures and pressures. Suitably, however, the oxygenate feed and olefinic co-feed are contacted with the formulated catalyst at a temperature in the range of from 200° C. to 650° C. In a further preferred embodiment the temperature is in the range of from 250° C. to 630° C., more preferably in the range of from 300° C. to 620° C., most preferably in the range of from 450° C. to 600° C. Preferably the reaction to produce the olefins is conducted at a temperature of more than 450° C., preferably at a temperature of 460° C. or higher, more preferably at a temperature of 490° C. or higher. At higher temperatures a higher activity and ethylene selectivity is observed. Aluminosilicates having one-dimensional 10-membered ring channels can be operated under oxygenate conversion conditions at such high temperatures with acceptable deactivation due to coking, contrary to aluminosilicates with smaller pores or channels, such as 8-membered ring channels. Temperatures referred to hereinabove represent reaction temperatures, and it will be understood that a reaction temperature can be an average of temperatures of various feed streams and the catalyst in the reaction zone.

In addition to the oxygenate, and the olefinic co-feed (when present), for example in the range of from 0.01 to 10 kg diluent per kg oxygenate feed, in particular from 0.5 to 5 kg/kg. Any diluent known by the skilled person to be suitable for such purpose can be used. Such diluent can for example be a paraffinic compound or mixture of compounds. Preferably, however, the diluent is an inert gas. The diluent can be argon, nitrogen, and/or steam. Of these, steam is the most preferred diluent. It can be preferred to operate with a minimum amount of diluent, such as less than 500 wt % of diluent based on the total amount of oxygenate feed, in particular less than 200 wt %, more in particular less than 100 wt %. Operation without a diluent is also possible.

The olefinic product or reaction effluent is typically fractionated. The skilled artisan knows how to separate a mixture of hydrocarbons into various fractions, and how to work up fractions further for desired properties and composition for further use. The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation. It is within the skill of the artisan to determine the correct conditions in a fractionation column to arrive at such a separation. He may choose the correct conditions based on, inter alia, fractionation temperature, pressure, trays, reflux and reboiler ratios.

At least a light olefinic fraction comprising ethylene and/or propylene and a heavier olefinic fraction comprising C4 olefins and less than 10 wt % of C5+ hydrocarbon species are normally obtained. Preferably also a water-rich fraction is obtained. Also a lighter fraction comprising methane, carbon monoxide, and/or carbon dioxide can be obtained, as well as one or more heavy fractions comprising C5+ hydrocarbons. Such a heavy fraction, that is not being recycled, can for example be used as gasoline blending component.

In a particular aspect the present invention provides a process for the preparation of an olefinic product, wherein use is made of the phosphorus treated catalyst of the present invention, which process comprises the step a) of reacting an oxygenate feedstock and an olefinic co-feed in a reactor in the presence of oxygenate conversion catalyst particles comprising both an aluminosilicate having one-dimensional 10-membered ring channels, and a molecular sieve having more-dimensional channels, to prepare an olefinic reaction effluent. Preferably the weight ratio between the one-dimensional molecular sieve and the more-dimensional molecular sieve is in the range of from 1:1 to 100:1. In a preferred embodiment, this process comprises the further steps of b) separating the olefinic reaction effluent into at least a first olefinic fraction and a second olefinic fraction; and c) recycling at least part of the second olefinic fraction obtained in step b) to step a) as olefinic co-feed; and d) recovering at least part of the first olefinic fraction obtained in step b) as olefinic product.

In step b) of this process according to the invention the olefinic reaction effluent of step a) is separated (fractionated). At least a first olefinic fraction and a second olefinic fraction, preferably containing C₄ olefins, are obtained. The first olefinic fraction typically is a light olefinic fraction comprising ethylene, and the second olefinic fraction is typically a heavier olefinic fraction comprising C4 olefins.

Preferably also a water-rich fraction is obtained. Also a lighter fraction comprising contaminants such as methane, carbon monoxide, and/or carbon dioxide can be obtained and withdrawn from the process, as well as one or more heavy fractions comprising C5+ hydrocarbons, including C5+ olefins. Such heavy fraction can for example be used as gasoline blending component. For example, the first olefinic fraction can comprise at least 50 wt %, preferably at least 80 wt %, of C1-C3 species, the recycled part of the second olefinic fraction can comprise at least 50 wt % of C₄ species, a heavier carbonaceous fraction that is withdrawn from the process can comprise at least 50 wt % of C₅₊ species.

In step c) at least part of the second olefinic fraction, preferably containing C₄ olefins, obtained in step b) is recycled to step a) as olefinic co-feed.

Only part of the second olefinic fraction or the complete second olefinic fraction may be recycled to step a).

In the process also a significant amount of propylene is normally produced. The propylene can form part of the light olefinic fraction comprising ethene, and which can suitably be further fractionated into various product components. Propylene can also form part of the heavier olefinic fraction comprising C4 olefins. The various fractions and streams referred to herein, in particular the recycle stream, can be obtained by fractionating in various stages, and also by blending streams obtained during the fractionation. Typically, an ethylene- and a propylene-rich stream of predetermined purity such as pipeline grade, polymer grade, chemical grade or export quality will be obtained from the process, and also a stream rich in C4 comprising C4 olefins and optionally C4 paraffins, such as an overhead stream from a debutaniser column receiving the bottom stream from a depropanizer column at their inlet. It shall be clear that the heavier olefinic fraction comprising C4 olefins, forming the recycle stream, can be composed from quantities of various fractionation streams. So, for example, some amount of a propylene-rich stream can be blended into a C4 olefin-rich stream. In a particular embodiment at least 90 wt % of the heavier olefinic fraction comprising C4 olefins can be formed by the overhead stream from a debutaniser column receiving the bottom stream from a depropanizer column at their inlet, more in particular at least 99 wt % or substantially all.

Suitably the olefinic reaction effluent comprises less than 10 wt %, preferably less than 5 wt %, more preferably less than 1 wt %, of C6-C8 aromatics, based on total hydrocarbons. Producing low amounts of aromatics is desired since any production of aromatics consumes oxygenate which is therefore not converted to lower olefins.

Any feature of any aspect of any invention or embodiment described herein may be combined with any feature of any aspect of any other invention or embodiment described herein mutatis mutandis.

Embodiments of the present invention will now be described by way of example only.

### EXAMPLE 1

A series of catalyst samples were prepared comprising 40 wt % zeolite (32 wt % ZSM-23 being a first molecular sieve and 8 wt % ZSM-5 being second molecular sieve), 36 wt % kaolin and 24 wt % silica, and to which and various amounts of phosphorus as detailed in the tables below. The samples were then catalytically tested and compared to samples without phosphorus.

In the samples ZSM-23 zeolite powder with a silica to alumina molar ratio (SAR) 46, and ZSM-5 zeolite powder with a SAR of 280 were used in the ammonium form in the weight ratio 80:20. The powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution.

Phosphorus was deposited on the catalyst by means of impregnation using acidic solutions containing phosphoric acid (H₃PO₄) or ammonium dihydrogen phosphate (NH₄H₂PO₄). The concentration of the solution was adjusted such to obtain the appropriate amount of phosphorus on the catalyst. After impregnation the catalysts were dried at 120° C. and were calcined at 600° C. for 2 hours. The phosphorus loading on the final catalysts is given in the Tables as wt % of elemental phosphorus in any phosphor species, based on the total formulated catalyst, and was determined by elemental analysis. The amount of phosphorus is based on the elemental weight of phosphorus (which does not need to be in elemental form though) and not on the total weight of phosphorus species present. This may be determined by elemental analysis and is also referred to as elemental phosphorus loading.

To test the samples for catalytic performance the respective catalyst powder was pressed into tablets and the tablets were broken into pieces and sieved. Dimethyl ether (DME) and 1-butene were reacted over the catalysts which were tested to determine their selectivity towards ethylene and propylene from oxygenates and their stability under such reaction conditions. For the catalytic testing, the sieve fraction of 40-60 mesh was used. Prior to reaction, the fresh catalyst in its ammonium-form was treated ex-situ in air at 600° C. for 2 hours.

The reaction was performed using a quartz reactor tube of 3.6 mm internal diameter. The catalyst samples were heated in argon to 525° C. and a mixture consisting of 3 vol % dimethyl ether, 3 vol % 1-butene, 1 vol % steam balanced in N₂ was passed over the catalyst at atmospheric pressure (1 bar). The Gas Hourly Space Velocity (GHSV) is determined by the total gas flow over the catalyst weight per unit time (ml·g_{catalyst}⁻¹·h⁻¹). The effluent from the reactor was analyzed by gas chromatography (GC) to determine the product composition. The composition has been calculated on a weight basis of all hydrocarbons analyzed. The selectivity has been defined by the division of the mass of product by the sum of the masses of all products.

Table 1 shows the results obtained for a first series of catalyst samples for impregnation with H₃PO₄, at various phosphorus concentrations, and for two times on stream. Cn refers to hydrocarbon species having n carbon atoms, Cn+ refers to hydrocarbon species having n or more carbon atoms (n being an integer) figures include all; Cn=refers to olefinic hydrocarbon species having n carbon atoms. The index sats refers to saturated carbon species, and tot or totals refer to all respective hydrocarbon species.

TABLE 1

| | Impregnation Compound | | | | | | | | | |
|---|---|---|---|---|---|---|---|---|---|---|
| | untreated | | | | | | | | | |
| | (comparative) | | H3PO4 | | H3PO4 | | H3PO4 | | H3PO4 | |
| | P-content (wt %) | | | | | | | | | |
| | 0 | | 0.4 | | 0.6 | | 0.8 | | 1 | |
| Time (h) | 1 | 8 | 1 | 8 | 1 | 8 | 1 | 8 | 1 | 8 |
| DME conversion (%) | 100 | 100 | 100 | 100 | 100 | 100 | 100 | 100 | 100 | 100 |
| C2-C5 (wt %) | 94.2 | 89.7 | 94.6 | 91.4 | 95.3 | 92.8 | 95.8 | 93 | 95.9 | 93.9 |
| C6+ (wt %) | 3.9 | 7.3 | 3.1 | 4.6 | 2.8 | 4.2 | 2.9 | 4.5 | 2.4 | 4 |
| C7-C8 aromatics (wt %) | 0.73 | 0.91 | 0.5 | 0.5 | 0.3 | 0.3 | 0.25 | 0.23 | 0.15 | 0.14 |
| C4 sats/C4 total wt/wt | 4.3 | 6.6 | 4.3 | 5.9 | 3.6 | 5.4 | 3.4 | 4.6 | 2.9 | 3.2 |
| DME breakthrough (h) | 24 | | 24 | | >25 | | >25 | | >25 | |
| GHSV-total (ml/g/h) | 24000 | | 24000 | | 24000 | | 24000 | | 24000 | |

| | Impregnation Compound | | | | | | | | | |
|---|---|---|---|---|---|---|---|---|---|---|
| | H3PO4 | | H3PO4 | | H3PO4 | | H3PO4 | | H3PO4 | |
| | P-content (wt %) | | | | | | | | | |
| | 1.2 | | 1.4 | | 1.6 | | 1.8 | | 2 | |
| Time (h) | 1 | 8 | 1 | 8 | 1 | 8 | 1 | 8 | 1 | 8 |
| DME conversion (%) | 100 | 100 | 100 | 100 | 100 | 100 | 100 | 100 | 100 | 100.00 |
| C2-C5 (wt %) | 95.2 | 93.1 | 94.4 | 92.6 | 93.8 | 92 | 93.5 | 90.5 | 92.7 | 90.1 |
| C6+ (wt %) | 3.2 | 5.5 | 4.2 | 6.3 | 4.8 | 6.7 | 5.4 | 8 | 6.3 | 8.6 |
| C7-C8 aromatics (wt %) | 0.1 | 0.08 | 0.08 | 0.1 | 0.02 | 0.02 | 0.02 | 0.02 | <0.02 | <0.02 |
| C4 sats/C4 total wt/wt | 2.4 | 2.4 | 2.2 | 2.2 | 1.6 | 2 | 1.6 | 2.4 | 1.4 | 2.6 |
| DME breakthrough (h) | 24 | | 25 | | 25 | | 20 | | 16 | |
| GHSV-total (ml/g/h) | 24000 | | 24000 | | 24000 | | 24000 | | 24000 | |

C2-C5 products include paraffins and olefins, and comprise at least 90 wt % of olefins.
C6+ excludes toluene, xylenes, ethylbenzene

As can be observed from Table 1, with increasing Phosphor content, the preferred product, C2-C5, does not decrease with increasing amount of phosphor and indicates that catalyst activity is sustained. As can be observed from the Table, with increasing phosphorus content, the make of unwanted by-products C7-C8 make and proportion of C4 saturates produced is gradually reduced. Another beneficial effect is that the ratio C4 saturates/C4 total is also decreased after treatment with phosphorus, and C4 saturates are unwanted by-products particularly when a C4 stream is to be recycled to the oxygenate conversion reaction. Moreover, the preferred product, C2-C5, also gradually increases, most pronounced in the range of from 0.1 to 1.8 wt % P, in particular of from 0.4-1.8 wt % P. Moreover the time until DME breakthrough was improved in the range of from 0.1 to 1.6 wt % P, in particular of from 0.4-1.6 wt % P.

Moreover in all cases in table 1 and 2, full conversion of DME is obtained.

TABLE 2

| Impregnation compound | NH4H2PO4 |
|---|---|
| P-content (wt %) | 1.4 |

| | | |
|---|---|---|
| Time (h) | 1 | 8 |
| DME conversion (%) | 100 | 100 |
| C2-C5 (wt %) | 92.6 | 89.7 |
| C6+ (wt %) | 6.2 | 8.4 |
| C7-C8 aromatics (wt %) | <0.02 | <0.02 |
| C4 sats/C4 total wt/wt | 2 | 3.2 |
| DME breakthrough (h) | 20 | |
| GHSV-total (ml/g/h) | 24000 | |

C2-C5 products include paraffins and olefins, and comprise at least 90 wt % of olefins. C6+ excludes toluene, xylenes, ethylbenzene

Table 2 shows one sample using an ammonium dihydrogen phosphate solution to deposit the phosphorus by impregnation. This sample also shows improved C2-C5 make and a reduction in C7-C8 aromatics and C4 saturates.

TABLE 3

| | Impregnation compound | | | | | |
|---|---|---|---|---|---|---|
| | | | | | untreated | |
| | H3PO4 | | NH4H2PO4 | | (comparative) | |
| | P-content (wt %) | | | | | |
| | 1 | | 1 | | 0 | |
| | Steam treated | | | | | |
| | 600 C. 5 h | | 600 C. 5 h | | 600 C. 5 h | |
| Time (h) | 1 | 8 | 1 | 8 | 1 | 8 |
| DME conversion (%) | 100 | 100 | 100 | 100 | 100 | 100 |
| C2-C5 (wt %) | 95.7 | 95.5 | 97.3 | 97.1 | 92.1 | 90.9 |
| C6+ (wt %) | 2.9 | 3 | 2.1 | 2.2 | 5 | 5.9 |
| C7-C8 aromatics (wt %) | 0.33 | 0.31 | 0.11 | 0.06 | 0.68 | 0.73 |
| C4 sats/C4 total (wt/wt) | 3.7 | 4 | 1.6 | 1.8 | 7.8 | 8.9 |
| GHSV-total (ml/g/h) | 6000 | | 6000 | | 6000 | |

C2-C5 products include paraffins and olefins, and comprise at least 90 wt % of olefins.
C6+ excludes toluene, xylenes, ethylbenzene

Table 3 shows the catalytic test results for catalyst samples along with a comparative sample that has not undergone phosphorus treatment; all samples after aging by steam treatment at 600 C for 5 hours prior to the catalytic testing. It can be seen from table 3 that the C2-C5 make is improved compared to the comparative example, and less by-products C6+, C7-C8, and C4 saturates are formed thus demonstrating a number of benefits for the samples in accordance with the invention compared to the comparative sample. Further experiments also showed significant reductions in the amount of coke produced when using a catalyst having a phosphorus containing compound on it.

### EXAMPLE 2

A formulated catalysts were prepared comprising 40 wt % zeolite (32 wt % ZSM-23 being a first molecular sieve and 8 wt % ZSM-5 being second molecular sieve), 36 wt % kaolin and 24 wt % silica, and to which various amounts of phosphorus are added as detailed in the tables below. The samples were then catalytically tested and compared to samples without phosphorus.

In the preparation of all the formulated catalyst, ZSM-23 zeolite powder with a silica to alumina molar ratio (SAR) 46, and ZSM-5 zeolite powder with a SAR of 280 were used in the ammonium form in the weight ratio 80:20. The powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution. The final formulated catalyst thus obtained is further referred to as catalyst 2A.

Starting from a sample of formulated catalyst 2A, phosphorus was deposited on the catalyst by means of impregnation using acidic solutions containing phosphoric acid (H₃PO₄). The concentration of the solution was adjusted such to deposit 1.5 wt % of phosphorous on the catalyst. After impregnation the catalysts were dried at 120° C. and were calcined at 600° C. for 2 hours. The phosphorous loading on the final catalysts is given in Table 4 as wt % of elemental phosphorous in any phosphor species, based on the total formulated catalyst, and was determined by elemental analysis. The amount of phosphorus is based on the elemental weight of phosphorus (which does not need to be in elemental form though) and not on the total weight of phosphorous species present. This may be determined by elemental analysis and is also referred to as elemental phosphorous loading. This formulated catalyst is further indicated as catalyst 2B.

Another formulated catalyst was prepared as described wherein above for catalyst 2A, with the exception that the ZSM-5 zeolite powder was first treated with phosphorus before mixing the catalyst with ZSM-23 and a binder before spray drying, resulting in a catalyst that has only one zeolite treated with phosphorus. Phosphorus was deposited on a ZSM-5 zeolite powder with a silica-to-alumina ratio of 280 by means of impregnation with an acidic solution containing phosphoric acid to obtain a ZSM-5 treated zeolite powder containing 0.6 wt % P. The ZSM-5 powder was calcined at 550° C. ZSM-23 with a silica-to-alumina ratio of 50 was mixed with the phosphorus treated ZSM-5 in a weight ratio of 80:20. The powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution. The obtained formulated catalyst is further referred to as example 2C.

Starting from a sample of formulated catalyst 2C, phosphorus was deposited on formulated catalyst 2C by means of impregnation using acidic solutions containing phosphoric acid (H₃PO₄) or ammonium dihydrogen phosphate (NH₄H₂PO₄). The concentration of the solution was adjusted such to deposit 1 wt % of phosphorous on the catalyst. After impregnation the catalysts were dried at 120° C. and were calcined at 600° C. for 2 hours. The obtained formulated catalyst is further referred to as example 2D

To test the four formulated catalysts 2A to 2D for catalytic performance the respective catalyst powder was pressed into tablets and the tablets were broken into pieces and sieved. Methanol and 1-butene were reacted over the catalysts which were tested to determine their selectivity towards olefins, mainly ethylene and propylene from oxygenates. For the catalytic testing, the sieve fraction of 60-80 mesh was used. Prior to reaction, the catalyst was treated ex-situ in air at 600° C. for 2 hours.

The reaction was performed using a quartz reactor tube of 1.8 mm internal diameter. The catalyst samples were heated in nitrogen to 525° C. and a mixture consisting of 3 vol % 1-butene, 6 vol % methanol, balanced in N₂ was passed over the catalyst at atmospheric pressure (1 bar). The Gas Hourly Space Velocity (GHSV) is determined by the total gas flow over the catalyst weight per unit time (ml·g_{catalyst}⁻¹·h⁻¹). The gas hourly space velocity used in the experiments was 24,000 (ml·g_{catalyst}⁻¹·h⁻¹). The effluent from the reactor was analyzed by gas chromatography (GC) to determine the product composition. The composition has been calculated on a weight basis of all hydrocarbons analyzed. The selectivity has been defined by the division of the mass of product by the sum of the masses of all products.

In Table 4, the results are indicated for the four catalyst 2A to 2D.

TABLE 4

| | Catalyst | | | | | | | |
|---|---|---|---|---|---|---|---|---|
| | 2A* | | 2B | | 2C* | | 2D | |
| P post treatment | No | | Yes | | No | | Yes | |
| Wt % P | 0 | | 1.5 | | 0.05 | | 1.05 | |
| Time (h) | 5 | 11 | 5 | 11 | 5 | 11 | 5 | 11 |
| DME conversion (%) | 100 | 100 | 100 | 100 | 100 | 100 | 100 | 100 |
| C2-C5 (wt %) | 91.9 | 88.6 | 93.4 | 91.3 | 94.2 | 90.7 | 91.7 | 88.4 |
| C6+ (wt %) | 6.8 | 10.2 | 6.4 | 8.5 | 4.6 | 7.2 | 7.8 | 11.0 |
| C7-C8 aromatics | 0.95 | 0.86 | 0.05 | 0.05 | 0.32 | 0.53 | 0.34 | 0.28 |
| (wt %) | | | | | | | | |
| C4 sats/C4 total | 8.1 | 9.3 | 2.4 | 3.1 | 5.5 | 7.7 | 2.9 | 5.4 |
| (wt/wt) | | | | | | | | |

| | | | | |
|---|---|---|---|---|
| GHSV-total | 24,000 | 24,000 | 24,000 | 24,000 |
| (ml/g/h) | | | | |

C2-C5 products include paraffins and olefins, and comprise at least 90 wt % of olefins.
C6+ excludes toluene, xylenes, ethylbenzene
*Not according to the invention.

It can be observed from the results obtained for catalyst 2A and 2B, that treating the formulated catalyst by depositing phosphor on the formulated catalyst has the beneficial effect that the ratio C4 saturates/C4 total is decreased after treatment with phosphorus, and C4 saturates are unwanted by-products particularly when a C4 stream is to be recycled to the oxygenate conversion reaction. It can further be observed that from the results obtained for catalyst 2C and 2D, that treating a formulated catalyst, of which one of the molecular sieves already comprised deposited phosphor due to a pre-treatment, by depositing phosphor subsequently to the formulation on the formulated catalyst again results in a decreased ratio of C4 saturates/C4 total.

Thus it is clear from the results, detailed above, that a post-treatment of the formulated catalyst by depositing phosphor on the formulated catalyst according to the invention provides an improved catalyst, comprising a phosphorus containing compound, for converting oxygenates to olefins.

1. A process for the manufacture of a formulated oxygenate conversion catalyst, the process comprising combining a first molecular sieve comprising aluminosilicate, a second molecular sieve, different from the first molecular sieve, the second molecular sieve having more-dimensional channels, and a matrix material; and,
treating the catalyst with a phosphorus containing compound after combination of the molecular sieves with the matrix material.

2. A process according to claim 1, wherein the phosphorus-containing compound is impregnated into the catalyst by impregnation with an aqueous solution of the phosphorus-containing compound.

3. A process according to claim 1, wherein the phosphorus-containing compound comprises at least one of PO₄³⁻, P—(OCH₃)₃ and P₂O₅.

4. A process according to claim 1, wherein the phosphorus-containing compound comprises at least one compound selected from the group consisting of ammonium phosphate, ammonium dihydrogen phosphate, dimethylphosphate, metaphosphoric acid and trimethyl phosphite.

5. A process according to claim 1, wherein the phosphorus containing compound is not a Group II metal phosphate.

6. A process according to claim 1, wherein the second molecular sieve is an aluminosilicate.

7. A process according to claim 1, wherein the channels of the second molecular sieve in at least one direction are 10-membered ring channels.

8. A process according to claim 6, wherein the second molecular sieve comprises MFI-type aluminosilicate and/or MEL-type aluminosilicate.

9. A process according to claim 1, wherein the first molecular sieve has one-dimensional 10-membered ring channels.

10. A formulated oxygenate conversion catalyst obtainable by a process according to claim 1, comprising:
a first molecular sieve comprising aluminosilicate;
a second molecular sieve, different from the first molecular sieve, the second molecular sieve having more-dimensional channels;
a matrix material;
wherein the catalyst comprises more of the first molecular sieve, than of the second molecular sieve, based on weight;
and wherein the formulated oxygenate conversion catalyst comprises a phosphorus or a phosphorus containing compound.

11. A catalyst according to claim 10, wherein the phosphorus is present as such or in a compound in an elemental amount of 0.05-10 wt % of the formulated catalyst.

12. A catalyst according to claim 10, wherein an external surface area of the formulated catalyst material is 1-500 m²/g.

13. A catalyst according to claim 10, comprising catalyst particles, wherein individual catalyst particles comprise both the first molecular sieve and the second molecular sieve.

14. A process for the preparation of an olefinic product in the presence of a catalyst as claimed in claim 1, the process comprising reacting an oxygenate feedstock in the presence of the catalyst to produce the olefinic product.

15. A process according to claim 14, wherein the oxygenate feedstock is reacted to produce the olefinic product in the presence of an olefinic co-feed.
